(19) United States
(12) Patent Application Publication
(10) Pub. No.: US 2019/0070167 A1
(43) Pub. Date: Mar. 7, 2019

(54) PITAVASTATIN CONTAINING PREPARATION AND METHOD FOR PRODUCING SAME

(71) Applicants: Imtiyaz Naikwadi, Pune (IN); Vikram Karne, Pune (IN); Prafulla Firodiya, Pune (IN); Makarand Avachat, Pune (IN); Sanjay C. Wagh, Pune (IN)

(72) Inventors: Imtiyaz Naikwadi, Pune (IN); Vikram Karne, Pune (IN); Prafulla Firodiya, Pune (IN); Makarand Avachat, Pune (IN); Sanjay C. Wagh, Pune (IN)

(21) Appl. No.: 15/698,135

(22) Filed: Sep. 7, 2017

(51) Int. Cl. A61K 31/4709; A61K 47/02; A61K 9/20

(52) U.S. Cl. CPC A61K 31/4709; A61K 9/2009; A61K 47/02

(57) ABSTRACT

The present invention provides a stable oral pharmaceutical composition comprising pitavastatin or a pharmaceutically acceptable salts thereof and at least one alkalizing agent selected from the group consisting of sodium hydroxide, sodium carbonate and sodium bicarbonate, and an aqueous solution or an aqueous dispersion of the pitavastatin containing preparation having a pH of more than 8 and 10 or less; and a method for producing the pitavastatin containing preparation.

### FIELD OF THE INVENTION

The present invention relates to stable oral pharmaceutical compositions comprising pitavastatin or pharmaceutically acceptable salts thereof and at least one alkalizing agent. The invention also provides the use of stable oral pharmaceutical compositions of pitavastatin for the treatment patients with hypercholesterolemia, familial hypercholesterolemia and the like.

### BACKGROUND OF THE INVENTION

Pitavastatin belongs to a well-known group of drugs, statins, which are useful for the treatment of hypercholesterolemia or hyperlipidemia. Statins block the hydroxyl-methylglutaryl-coenzyme (HMG-CoA) reductase, thereby specifically inhibiting cholesterol synthesis in the liver. Although cholesterol is a vital component of all cells, it also contributes to plaque formation in arteries, which increases the risk of high blood pressure, heart attack and stroke. Statins stabilize the plaques, making them less prone to rupturing and subsequently forming a dangerous blood clot.

Generally pharmaceutical compositions which are unstable in an acidic or basic environment may require a basic or acidic excipient to enhance storage stability. However, the active ingredient in the compositions may comprise functional groups that are sensitive to both acidic and basic environments and neutral environments may be needed.

Pitavastatin is susceptible to heat, moisture, low pH environment and light. In an acidic environment, the hydroxy acid moiety present in pitavastatin converts to lactone and various isomers. So during formulation and storage, pitavastatin may be further destabilized by contact with the molecular moieties of other excipients. Since commonly used pharmaceutical excipients, such as binders, diluents, anti-adherents and surfactants may adversely interact with pitavastatin, it is essential to stabilize pitavastatin containing preparations by the addition of a stabilizer. As the stability of Pitavastatin containing preparations is poor, there is a possibility that efficacy of the drug is decreased and safety is impaired. Accordingly, it is necessary to stabilize pitavastatin or a salt thereof in the pitavastatin containing preparations.

U.S. Pat. No. 5,030,447 discloses stabilization method of HMG-CoA reductase inhibitor-containing preparation, a method of blending a basifying agent which gives a pH of 9 or more with an aqueous dispersion of a pravastatin-containing preparation.

U.S. Pat. No. 5,356,896 discloses stabilization method of fluvastatin sodium containing preparations, a method of blending a basifying agent which gives a pH of at least 8 with an aqueous solution or a dispersion of preparation. The basifying agent used is a combination of sodium bicarbonate and calcium carbonate.

U.S. Pat. No. 9,399,064 discloses stabilization of pitavastatin containing preparation, a method of blending a basic additive selected from the group consisting of magnesium hydroxide, magnesium carbonate, and magnesium silicate, wherein an aqueous solution or an aqueous dispersion of the pitavastatin containing preparation has a pH of more than 8 and 10 or less.

U.S. Patent Publication No. US 2013/0310420 A1 discloses stabilization of pitavastatin containing preparation, a method of blending magnesium oxide, wherein an aqueous solution or dispersion has pH of from 10 to 10.8. The composition does not include pH regulators.

The prior art references U.S. Pat. No. 5,356,896 provide no specific description or method for stabilization of pitavastatin containing preparation. U.S. Pat. No. 9,399,064 and US 2013/0310420 discloses use of specific magnesium compounds as stabilizers and there is no disclosure or teaching in the art about the method to develop stable formulations of pitavastatin without employing specific magnesium compounds as stabilizers, which can also exhibit rapid or modified disintegration as well as equivalent in vitro (dissolution) profile over a wide dose range.

The prior art references teach that since pitavastatin is unstable at low pH environment, it should be formulated with a basic agent or a substance which maintains the pH to alkaline and mentions several basic agents.

But pitavastatin salts are still unstable at a higher pH range and the outward appearance changes with time. This emphasizes that there is a need in the art for a simple and more stable formulation of pitavastatin at a greater pH range.

The applicants of the present invention found that Pitavastatin sodium salt is unstable even in a basic environment after the use of magnesium containing basic agents and other known basic agents in the prior art. They unexpectedly found that the compositions of pitavastatin sodium salt are stable when stabilized with sodium bicarbonate. Hence there still remains a need to develop an alternative stable oral pharmaceutical compositions of pitavastatin in order to achieve desired dissolution profile of the formulations containing wide dose of active ingredient. Such a formulation will prevent degradation of pitavastatin.

### SUMMARY OF THE INVENTION

It is an objective of the invention to develop a robust, stable and acceptable oral pharmaceutical formulation of pitavastatin and pharmaceutically acceptable salts thereof.

Some aspects of the disclosure relate to a stable oral pharmaceutical composition comprising pitavastatin or salts thereof, and at least an alkalizing agent selected from the group consisting of sodium hydroxide, sodium carbonate and sodium bicarbonate and one or more pharmaceutically acceptable excipients, wherein an aqueous solution or an aqueous dispersion of the pitavastatin-containing preparation has a pH of more than 8 and 10 or less.

Some aspects of the disclosure relate to a solid dosage form comprising pitavastatin or pharmaceutically acceptable salts thereof, and at least one alkalizing agent selected from the group consisting of sodium hydroxide, sodium carbonate and sodium bicarbonate and one or more pharmaceutically acceptable excipients, wherein an aqueous solution or an aqueous dispersion of the pitavastatin-containing preparation has a pH of more than 8 and 10 or less.

Some aspects of the disclosure relate to a stable oral pharmaceutical composition comprising pitavastatin or pharmaceutically acceptable salts thereof, and at least one alkalizing agent selected from the group consisting of sodium hydroxide, sodium carbonate and sodium bicarbonate and one or more pharmaceutically acceptable excipients, wherein the composition retains at least 80% of the potency of pitavastatin or salts thereof in the pharmaceutical composition after storage at 40° C. and 75% relative humidity for six months.

Some aspects of the disclosure relate to a process for preparing a stable oral pharmaceutical composition of pitavastatin or salts thereof. The process includes the steps of admixing, granulating and/or coating pitavastatin or salts thereof with at least one alkalizer selected from the group consisting of sodium hydroxide, sodium carbonate and sodium bicarbonate.

Some aspects of the disclosure relate to a method of treating hypercholesterolemia, familial hypercholesterolemia and the like in patient comprising administering to said subject a stable oral pharmaceutical composition comprising pitavastatin or salts thereof, at least one alkalizing agent selected from the group consisting of sodium hydroxide, sodium carbonate and sodium bicarbonate and one or more pharmaceutically acceptable excipients.

### DETAILED DESCRIPTION OF THE INVENTION

The present disclosure relates to stable oral pharmaceutical compositions comprising pitavastatin or pharmaceutically acceptable salts thereof and at least one alkalizing agent selected from the group consisting of sodium bicarbonate, sodium carbonate and sodium hydroxide.

As used herein the term “stable” is understood to mean that the pharmaceutical composition of pitavastatin is stable when subjected to the stability conditions of 40° C. and 75% RH for 6 months, wherein the total degradation product is not more than 1%.

As used herein “pitavastatin” encompasses free base, pharmaceutically acceptable salts, pharmacologically active metabolites of pitavastatin and their pharmaceutically acceptable salts, hydrates, its enantiomers or its racemates unless otherwise noted. Pitavastatin is also known by the names NK-104, Itavastatin and Nisvastatin.

Pitavastatin calcium is known by the chemical name: (3R,5S)-7-[2-cyclopropyl-4-(4-fluorophenyl)quinolin-3-yl]-3,5-dihydroxy-6-(E)-heptenoic acid hemicalcium salt. The amount of pitavastatin or pharmaceutically acceptable salts thereof to be used ranges from about 1 mg to about 4 mg.

Pitavastatin sodium is known by the chemical name: Sodium (3R, 5S, E)-7-(2-cyclopropyl-4-(4-fluorophenyl)-quinolin-3-yl)-3, 5-dihydroxyhept-6-enoate. The amount of pitavastatin or pharmaceutically acceptable salts thereof to be used ranges from about 1 mg to about 4 mg.

As used herein ‘pharmaceutically acceptable salts’ include but are not limited to salts of sodium, calcium, magnesium and potassium or esters such as ethyl esters or methyl esters of these. Preferably, pitavastatin is used as pitavastatin sodium.

As used herein the terms ‘alkalizing agent’, ‘stabilizer’, ‘alkalizer’ and ‘basifying agent’ shall refer to one or more pharmaceutically acceptable substances capable of imparting a pH of more than 8 and 10 or less to an aqueous solution or dispersion of the composition of the invention. Alkalizing agents are used to stabilize the composition containing pitavastatin by creating a ‘micro-pH’ around the particles of the composition when water is adsorbed. Examples can be suitable inorganic bases which include (but are not limited to) alkali metal carbonates (such as sodium carbonate, potassium carbonate, calcium carbonate and the like), alkali earth metal bicarbonates (such as sodium bicarbonate, potassium bicarbonate, magnesium bicarbonate and the like). The amount of alkalizing agent can range from about 2% to about 70% by weight of the composition, preferably from about 2% to about 50% by weight of the composition and more preferably from about 4% to about 10% by weight of the composition. The alkalizing agent may be added to the composition in such defined amounts that is necessary for making the aqueous solution or dispersion of the composition having pH of more than 8 and 10 or less.

The pH as referred to herein indicates the pH value to be determined in such a manner that a unit dose of a solid preparation comprising pitavastatin or its salt or ester is sampled and dissolved or dispersed in from 1 to 10 ml of pure water, and the pH of the resulting aqueous solution or dispersion is measured.

The pharmaceutical composition of the present invention can be formulated into various dosage forms. The preferred dosage is oral solid preparations. For example, the composition may be formulated into tablets, granules, powders, troches, capsules, chewable, film-coated preparations of these and/or sugar-coated preparations thereof.

But the pharmaceutical composition of the present invention is not limited to such oral dosage forms and can be given through other ways like parenteral administration, suppository, transdermal preparations etc.

Where the pharmaceutical composition of the present invention is formulated into such peroral solid preparations, any of vehicles (excipients), binders, disintegrators and lubricants can be added thereto, if desired.

The pharmaceutical compositions may be prepared by conventional techniques and is not restricted to, dry granulation, wet granulation, melt granulation, direct compression, extrusion-spheronization or compression coating. As pitavastatin sodium salt is coarser and it contains high water content (15.2 to 21.1%). It is preferred to adopt wet granulation approach.

The pharmaceutically acceptable excipients include but are not limited to diluents, disintegrants, binders, solubility enhancing agents, lubricants and glidants known to person skilled in the art.

Diluent may be any pharmaceutically acceptable, non-toxic diluent. Examples of diluents include but are not limited to lactose, microcrystalline cellulose, starch, calcium hydrogen phosphate, mannitol, fructose, dextrose, sucrose, maltose and the like.

Suitable disintegrant(s) include one or more of crospovidone, low-substituted hydroxypropyl cellulose, croscarmellose sodium, sodium starch glycolate, starch, carmellose calcium and the like.

Suitable binders include one or more of polyvinylpyrrolidone, low-substituted hydroxypropyl methyl cellulose, hydroxypropyl cellulose, starch, sugar, gums and the like.

Suitable solubility enhancing agents include one or more surfactants. The surfactants may be any of the known pharmaceutically acceptable surfactants, including nonionic, anionic and cationic surfactants.

Suitable lubricants include one or more of magnesium stearate, stearic acid, calcium stearate, sodium stearyl fumarate, sodium benzoate or the like.

Suitable glidants include one or more of colloidal silicon dioxide, talc or the like.

In some embodiments, stable oral pharmaceutical compositions may be in the form of matrix comprising pitavastatin or pharmaceutically acceptable salts thereof, alkalizing agents and suitable pharmaceutically acceptable excipient and optionally coated with film coating agent.

In some embodiments, the present disclosure relates to oral stable pharmaceutical compositions comprising pitavastatin or pharmaceutically acceptable salts thereof, and an alkalizing agent and optionally pharmaceutically acceptable excipients.

In some embodiments, stable oral pharmaceutical compositions may be formulated in the form of matrix system wherein pitavastatin is dissolved and/or dispersed in a matrix system, which includes pitavastatin and alkalizing agents.

Suitable solvents used for granulation include one or more of water, ethyl alcohol, isopropyl alcohol, acetone, and mixtures thereof.

In some embodiments, the pharmaceutical compositions of the invention may optionally be coated with a functional and/or nonfunctional coating. The coating includes one or more coating additives, such as film forming polymers, plasticizers, coloring agents, opacifiers, solvents and lubricants/glidants.

Suitable film-forming polymers include one or more of ethyl cellulose, hydroxypropyl methylcellulose, methylcellulose, carboxy methylcellulose, hydroxymethyl cellulose, hydroxypropyl methylcellulose phthalate, cellulose acetate phthalate, cellulose acetate trimellitate; waxes such as polyethylene glycol; methacrylic acid polymers, such as Eudragit® RL and RS; and gums, such as xanthan gum. Alternatively, commercially available coating compositions comprising film-forming polymers marketed under various trade names, such as Opadry® may also be used for coating.

A polymer solution or dispersion may be prepared in various solvents, including one or more of water, ethanol, isopropyl alcohol, acetone, ether, or mixtures thereof.

Suitable plasticizers include one or more of acetylated triacetin, triethyl citrate, tributyl citrate, glycerol tributyrate, diacetylatedmonoglyceride, polyethylene glycols, propylene glycol, sesame oil, acetyl tributyl citrate, acetyl triethyl citrate, diethyl oxalate, diethyl phthalate, diethyl maleate, diethyl fumarate, dibutyl succinate, diethylmalonate, dioctyl phthalate and dibutyl sebacate. Suitable opacifiers include titanium dioxide.

Suitable coloring agents include one or more of Iron Oxide, Ferric Oxide Yellow, Lake of Tartrazine, Allura Red, Lake of Quinoline Yellow and Lake of Erythrosine.

The composition may be coated using techniques such as spray coating in a conventional coating pan, fluidized bed processor or dip coating.

In some embodiments, the pharmaceutical composition of the present invention may be produced by wet granulation technique where initially some amount of excipients and alkalizer are mixed. Pitavastatin and binder are dissolved in water and used as granulation aid. Further the granules formed are dried, mixed with lubricants and compressed into tablets.

### BEST MODES OF CARRYING OUT THE INVENTION

Examples of the pharmaceutical composition of the present invention are mentioned below, which, however, are not intended to restrict the scope of the invention.

TABLE 1
Example 1, without alkalizing agent.

| | | |
|---|---|---|
| S. No | Ingredient | Qty (mg/Tab) |

Drug core

| | | |
|---|---|---|
| 1 | pitavastatin sodium (eq. to pitavastatin)* | 4.206 (5.245) |
| 2 | Lactose monohydrate | 268.594 (267.594) |
| 3 | Low-substituted Hydroxypropyl cellulose | 38.4 |
| 4 | Hypromellose | 6.400 |
| 5 | Purified Water | q.s |
| 7 | Magnesium Stearate | 2.400 |

| | |
|---|---|
| Weight of core Tablet | 320.000 |

Film Coating

| | | |
|---|---|---|
| 8 | Film coating | 9.600 |
| 9 | Purified water USP*** | Q.S. |

| | |
|---|---|
| Total Film Coated Tablet Weight | 329.600 |

### Brief Manufacturing Procedure:

1. Weighed quantity of Hydroxypropyl cellulose along with Hydroxypropyl Methylcellulose and lactose is sifted through Sieve.
2. The blend of step 1 is loaded in Rapid Mixer Granulator and mixed for 10 minutes.
3. Weighed quantity of pitavastatin sodium and Hydroxypropyl methylcellulose are dissolved in water.
4. The blend of step 2 is granulated using purified water containing pitavastatin sodium and Hydroxypropyl methylcellulose.
5. The wet granules of step 4 are dried in Air dryer
6. The dried granules of step 4 are passed through Sieve.
7. The extra granular quantity of Hydroxypropyl cellulose is passed through Sieve.
8. The granules of step 6 are mixed with Hydroxypropyl cellulose of step 7.
9. The blend of step 8 is lubricated with Magnesium Stearate
10. The lubricated blend of step-9 is compressed into tablets using suitable size and shape punch.
11. The compressed tablets of step 10 are coated using

### Example 2

TABLE 2
Example 2, with sodium bicarbonate as alkalizing agent.

| | | |
|---|---|---|
| S. No | Ingredient | Qty (mg/Tab) |

Drug core

| | | |
|---|---|---|
| 1 | pitavastatin sodium (eq. to | 4.206 (5.104) |
| | pitavastatin)* | |
| 2 | Lactose monohydrate | 248.594 (247.696) |
| 3 | Low-substituted Hydroxypropyl cellulose | 38.4 |
| 4 | Hypromellose | 6.400 |
| 5 | Sodium bicarbonate | 20.000 |
| 6 | Purified Water | q.s |
| 8 | Magnesium Stearate | 4.8 |

| | |
|---|---|
| Weight of core Tablet | 322.400 |

Film Coating

| | | |
|---|---|---|
| 9 | Film coating | 9.600 |
| 10 | Purified water USP*** | Q.S. |

| | |
|---|---|
| Total Film Coated Tablet Weight | 332.000 |

Brief Manufacturing Procedure:

1. Weighed quantity of Hydroxypropyl cellulose along with Hydroxypropyl Methylcellulose, Sodium bicarbonate and lactose is sifted through Sieve.
2. The blend of step 1 is loaded in Rapid Mixer Granulator and mixed
3. Weighed quantity of pitavastatin sodium and Hydroxypropyl methylcellulose are dissolved in water.
4. The blend of step 2 is granulated using purified water containing pitavastatin sodium and Hydroxypropyl methylcellulose.
5. The wet granules of step 4 are dried in Air dryer
6. The dried granules of step 4 are passed through Sieve.
7. The extra granular quantity of Hydroxypropyl cellulose is passed through Sieve.
8. The granules of step 6 are mixed with Hydroxypropyl cellulose of step 7
9. The blend of step 8 is lubricated with is lubricated with Magnesium Stearate
10. The lubricated blend of step-9 is compressed into tablets using suitable size and shape punch.
11. The compressed tablets of step 10 are coated

### Example 3

TABLE 3
Example 3, with sodium hydroxide as alkalizing agent.

| | | |
|---|---|---|
| S. No | Ingredient | Qty (mg/Tab) |

Drug core

| | | |
|---|---|---|
| 1 | pitavastatin sodium (eq. to | 4.206 (5.154) |
| | pitavastatin)* | |
| 2 | Lactose monohydrate | 268.194 (267.246) |
| 3 | Low-substituted Hydroxypropyl cellulose | 38.4 |
| 4 | Hypromellose | 6.400 |
| 5 | Sodium hydroxide | 0.4 |
| 6 | Purified Water | q.s |
| 8 | Magnesium Stearate | 2.4 |

| | |
|---|---|
| Weight of core Tablet | 322.400 |

Film Coating

| | | |
|---|---|---|
| 9 | Film coating | 9.600 |
| 10 | Purified water USP*** | Q.S. |

| | |
|---|---|
| Total Film Coated Tablet Weight | 332.600 |

Example 2 was packed in HDPE pack, which was then subjected to stability studies at 40° C. and 75% RH for six months.

Assay determination was carried out using HPLC method involving C-18 column and mobile phase comprising a mixture of buffer and acetonitrile in the ratio of 700:300.

Relative substance (RS) determination was carried out using a HPLC method involving C-18 column and mobile phase comprising a mixture of Mobile Phases A and B, the composition of which is given below:

Buffer—Ammonium acetate in water.
Mobile Phase A—Mixture of buffer and acetonitrile in the ratio of 800:200.
Mobile Phase B—Mixture of buffer, tetrahydrofuran and acetonitrile in the ratio of 200:200:600.

TABLE 4
Stability Data of a Pharmaceutical Composition
of pitavastatin as per Example 2

| | Initial | 6 Months (HDPE Pack) |
|---|---|---|
| Assay (%) | 101.9 | 105.2 |
| Total Degradation product | 0.15 | 0.32 |

The initial and six months samples were analyzed for assay and total degradation product. The pharmaceutical composition was found to be stable with regard to the total degradation product and assay as given in Table 4.

1. A stable oral pharmaceutical composition comprising pitavastatin sodium and an alkalizing agent selected from the group consisting of sodium hydroxide, sodium carbonate and sodium bicarbonate and pharmaceutically acceptable excipients, wherein an aqueous solution or an aqueous dispersion of the pitavastatin sodium composition has a pH of more than 8 and 10 or less.

2. (canceled)

3. (canceled)

4. The pharmaceutical composition of claim 1, wherein an amount of alkalizing agent range is from about 2% to about 50% by weight of the composition.

5. The pharmaceutical composition of claim 1, wherein an amount of alkalizing agent range is from about 4% to about 10% by weight of the composition.

6. (canceled)

7. The pharmaceutical composition of claim 1, wherein the composition is prepared by blending at least one alkalizing agent with pitavastatin sodium, wherein an aqueous solution or an aqueous dispersion of said preparation has a pH of more than 8 and 10 or less.

8. The pharmaceutical composition of claim 1, wherein the composition retains at least 80% of a potency of pitavastatin or salts thereof in the pharmaceutical composition after storage at 40° C. and 75% relative humidity for six months.
